(19) United States
(12) Patent Application Publication
(10) Pub. No.: US 2011/0196432 A1
(43) Pub. Date: Aug. 11, 2011

(54) METHOD AND SYSTEM FOR COMPRESSION AND FIXATION OF TENDON TO BONE

(76) Inventor: Jack C. Griffis, III, Decatur, GA (US)

(21) Appl. No.: 13/016,872

(22) Filed: Jan. 28, 2011

Related U.S. Application Data
(60) Provisional application No. 61/299,977, filed on Jan. 30, 2010.

(51) Int. Cl. A61B 17/16; A61F 2/08

(52) U.S. Cl. 606/86 R; 623/13.14

(57) ABSTRACT

Methods and devices may be used to compress and fix soft tissue to bone. Exemplary embodiments described including methods of fixing tendon to bone in an anterior cruciate ligament replacement surgery. Through compressing tendon in situ within bone, tendons may be fixed to bone while limiting axial movement of tendons and adverse effects on tendon tension.

### CROSS REFERENCE TO RELATED APPLICATIONS

This present application claims the benefit of Provisional U.S. Application Ser. No. 61/299,977, filed Jan. 30, 2010 and entitled “METHOD AND SYSTEM FOR COMPRESSION AND FIXATION OF TENDON TO BONE,” the disclosure of which is incorporated herein by reference.

### SUMMARY

Methods and devices may be used to compress and fix soft tissue to bone. Exemplary embodiments described including methods of fixing tendon to bone in an anterior cruciate ligament replacement surgery. Through compressing tendon in situ within bone, tendons may be fixed to bone while limiting axial movement of tendons and adverse effects on tendon tension.

### A BRIEF DESCRIPTION OF THE DRAWINGS

FIG. 1 shows simplified schematic of a human knee.

FIG. 2 is an example of bone tunnel positions for performing an ACL repair.

FIG. 3 is a flowchart of an embodiment of a surgical method for compression and fixation of tendon to bone.

FIG. 4 are experimental results of straining tendon under compressive stress at particular rates of strain.

FIG. 5 is a schematic of a front view of an exemplary embodiment of a tendon bundle to be fixated on the upper extremity of the tibia in an embodiment of an ACL repair.

FIG. 6 is a schematic of a lateral view of an exemplary embodiment of a tendon bundle to be fixated on the upper extremity of the tibia in an embodiment of an ACL repair.

FIG. 7A is schematic front view of a tendon bundle in a bone, showing the placement of a cross-section A-A.

FIG. 7B is a schematic cross-section A-A view of the tendon bundle in the bone.

FIG. 8 is a perspective schematic view of an exemplary embodiment of an expandable member being inserted into the bone with the tendon bundle.

FIG. 9 is a schematic cross-section A-A view of the expandable member being inserted into the bone.

FIG. 10 is a schematic cross-section A-A view of the expandable member positioned within the bone and contacting the tendons of the tendon bundle.

FIG. 11 is a schematic cross-section A-A view of the expandable member in an exemplary expanded state.

FIG. 12 is a schematic cross-section A-A view of the bone and tendon bundle after the expandable member has been retracted from its exemplary expanded state and removed from the bone.

### DETAILED DESCRIPTION

The following description of various embodiments is merely exemplary in nature. While various embodiments have been described for purposes of this specification, various changes and modifications may be made to the embodiments disclosed herein.

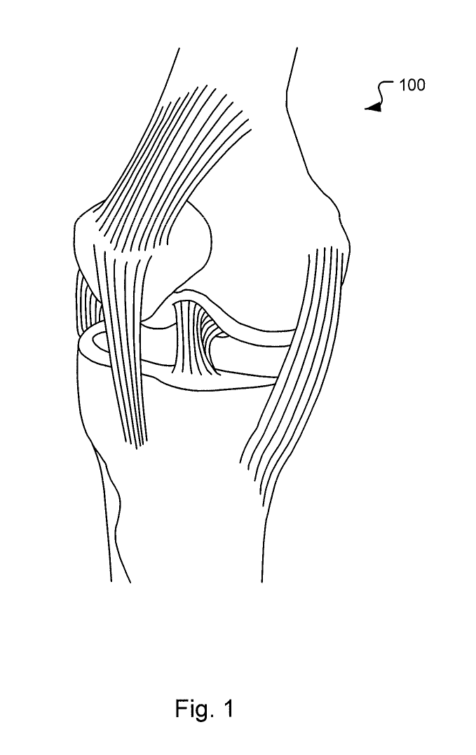

FIG. 1 shows an overview of the general layout of tendons in a human knee structure 100. Shown therein are the patella, the femur and the tibia together with supporting tendons, including the anterior cruciate ligament (ACL). Injuries to the ACL are some of the most common form of knee injuries. Embodiments described herein with references to the ACL or other parts of the knee are exemplary embodiments only as they may serve to generally orient the description with respect to a particular surgery. The techniques and devices described herein may also be used in other surgeries for attaching soft tissue to bone and the techniques described herein may be readily adapted to those other surgeries by surgeons skilled in the art.

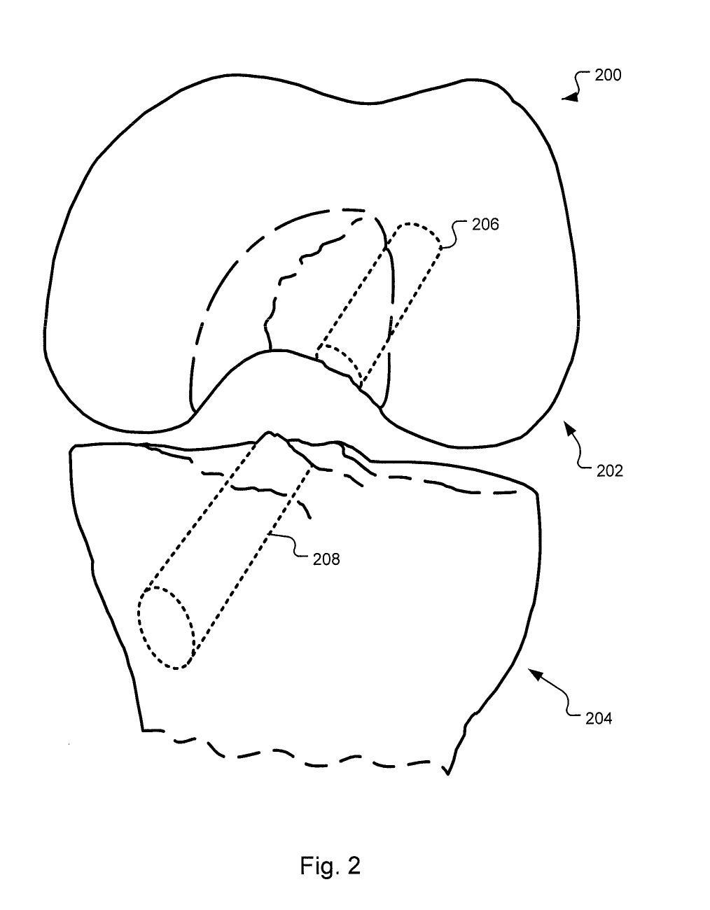

FIG. 2 shows another view of a knee joint 200 with an exemplary positioning of a tunnel in the tibia and femur into which tendons or other grafts of soft tissue may be inserted and fixed to the bone in order to repair torn and/or replace damaged ligaments in the knee. In the exemplary bone tunnels 206 and 208, cavities are prepared in the femur 202 and the tibia 204 where soft tissue (e.g., tendon) may be fixed to both bones and serve as a replacement to a patient's original ACL. The replacement soft tissue may be anchored in the femur 202 and passed through the tibia 204 to create the replacement ligament. Tension may then be applied to the replacement ligament across the knee joint to an appropriate level of tension against the fixed end of the replacement ligament in the femur 202. Thereafter, the replacement ligament may be fixed to the tibia 204 thereby completing the installation of the replacement ligament between the femur and tibia.

The description herein describes particular systems and methods for attaching soft tissue (e.g., tendon) to bone. The systems and methods described herein may be used in other surgeries and for other purposes as readily adaptable by surgeons with skill in the art.

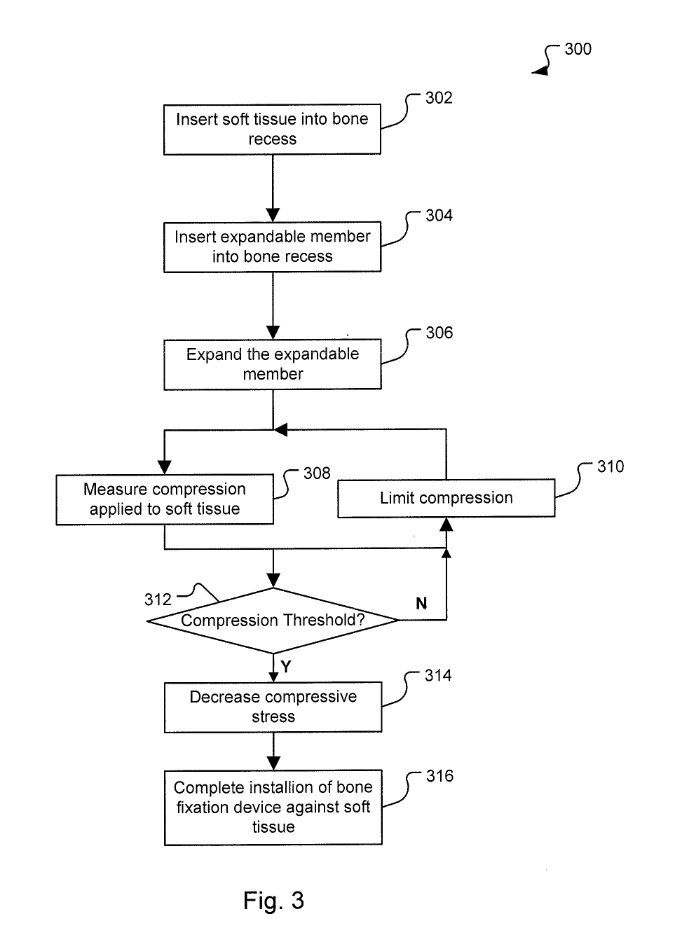

FIG. 3 shows an exemplary method 300 for attaching soft tissue to bone. The method is useful for attaching human or animal tissues (e.g., tendon, ligament) to bone, however, other tissues and materials may be substituted by those skilled in the art. The method includes inserting soft tissues into a bone recess 302 and inserting an expandable member into the bone recess 304. Steps 302 and 304 may be performed simultaneously or in sequence and may be performed in any order. Steps 302 and 304 may be performed with the aid of a guide wire, or may be performed generally as understood by those skilled in the art. The expandable member is then expanded 306 within the bone recess with at least a portion of the expandable member pressing the tendon against an inner surface of the bone recess. The expansion 306 of the expandable member thereby applies compressive forces to the soft tissue. The compression of the soft tissue is measured 308 and may be limited 310 based on the measurement. There are several embodiments through which compression may be measured 308 and limited 310.

In one embodiment, compression of soft tissue may be measured 308 through measuring some attribute of the expandable member. For example, if the expandable member contains an inflatable body that is inflated with some fluid, the pressure of the fluid may be measured and the compression of the soft tissue may thereby be measured 308 through calculation. In another embodiment, an expansion control device that mates with the expandable member may be mated with the expandable member at a measured extent and/or a measured rate. In this example, the measured extent and/or measured rate of the mating of the expansion device with the expandable member may be converted into a measured compression (step 308) through calculation.

In one embodiment, a balloon catheter may be used as an expandable member, possibly with modifications as described further herein. For example, a balloon catheter such as may be used for angioplasty may be coupled with an expansion control device for the balloon catheter. In another embodiment, techniques for manufacturing a balloon catheter and related expansion control device may be adapted for use in the methods and systems described herein. For example, the expandable member and expansion control device of a balloon catheter system, and techniques for making the same, may be adapted for use with tendon fixation such as, for example, modifying the pressures attainable by the system, modifying/adding exterior surface features of the expandable member, and/or constructing the expandable member to accept a reinforcing structure, as described further herein.

Measuring 308 and/or calculations may include information about the characteristics of the expandable element, information about manipulating/expanding 306 the expandable element and/or information about the bone and/or soft tissue.

Compression of the soft tissue may be limited 310 in several ways. For example, the fluid used to inflate an inflatable body of an expandable member may have a limited pressure that thereby limits compression 310 of the soft tissue. As another example, the mating of elements may be performed to a limited extent and/or a limited rate in order to limit compression 310.

While compression applied to the soft tissue is measured 308, a determination may be made whether a compression threshold (e.g., strain threshold, stress threshold) of the soft tissue has been met 312. As described further herein, this compression threshold may be based on biologically-relevant data of the soft tissue, and may depend on the soft tissue selected. The determination of whether the compression threshold has been met 312 may be made in any of the manners described for measuring compression 308 on the soft tissue. The strain imparted to the soft tissue may therefore be measured directly or indirectly through measuring the extent that the expandable member may be deemed to have expanded through calculations performed on other measured properties of the expandable member. In another embodiment, a compression threshold may be determined to have been met 312 based on an increase or decrease in a measured value (e.g., pressure) in response to an increase or decrease of a constraint applied to the expandable member (e.g., fluid inserted into an inflatable body). Indirect measurements of whether the compression threshold has been met 312 may include calculations based on information known about the soft tissue as described further herein.

Calculations may be used to limit compression 310 or determine a compression threshold 312 in similar manners to how compression is measured 308. Based on those calculations (which may be performed before the surgical methods herein), guides, instruments and/or device calibrations may be developed to use/contain information for these calculations or may perform part/all of the calculations. Those guides, instruments and/or device calibrations may be developed and used in a surgical method to perform or aid a surgeon in performing the measuring 308, limiting 310 steps, as well as determining whether a compression threshold has been met 312.

After a compression threshold has been determined to have been met 312 the method may include the optional step of decreasing the compressive stress 314 on the soft tissue. In one embodiment, decreasing compressive stress 314 may be performed through controlling the expandable member to stop or reverse the step 306 of expanding the expandable member. For example, the compressive stress on the soft tissue may be decreased 314 through removing fluid from an inflatable body of the expandable member. As another example, an expansion control device used to expand the expandable member may be unmated from the expandable member.

A bone fixation device may be installed 316 after a determination is made 312 that a compression threshold has been met and optionally after compressive stress on the soft tissue has been decreased 314, as described further herein. Installation of the bone fixation device may be completed 316 using several embodiments of a bone fixation device, some of which may include the expandable member used to provide the compressive stress to the soft tissue. The embodiments of the bone fixation device are described further herein and particularly with respect to the figures illustrating bundles of soft tissue and bone.

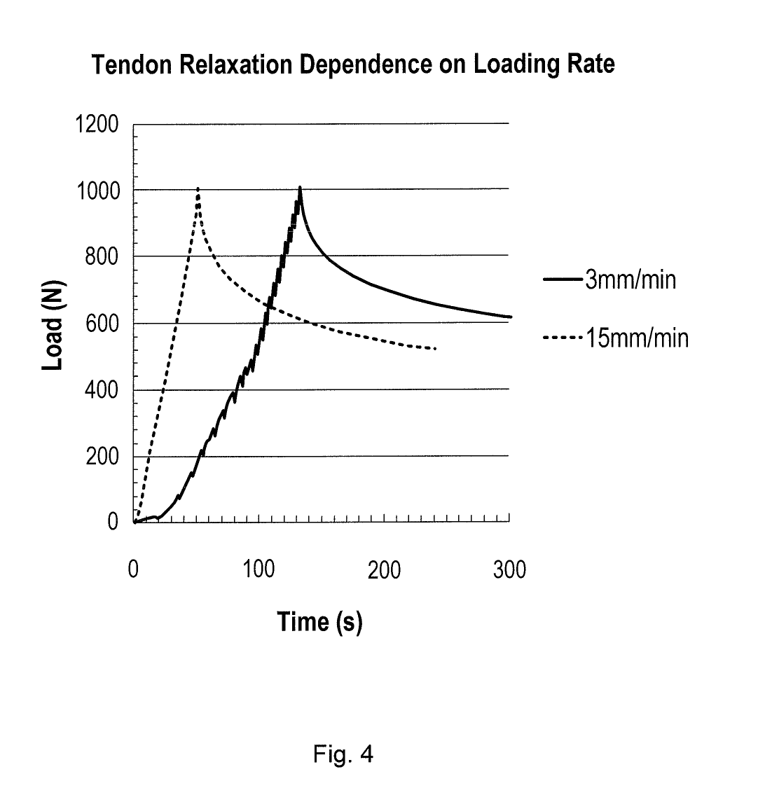

FIG. 4 shows experimental data for exemplary compressions of tendon grafts. The grafts were compressed at different rates of strain (e.g., mm/min) and exhibited the responsive stresses shown in FIG. 3 during the time period of compression indicated on the horizontal axis. Behaviors characteristic of different soft tissues may be similarly measured, extrapolated and otherwise used in support of the methods and systems described herein.

Based on experimental data collected about the soft tissue used in the surgical procedure, a compression threshold may be determined for the soft tissue. In the example of a tendon used as a soft tissue, there may be a compression threshold set to a point where additional compressive stress on the tendon does not create a significant strain response from the tendon. For example, whereas a tendon may respond to a compressive stress with a particular rate of strain adaptation over a period of time, that strain adaptation may change or the rate may change (e.g., a zero-crossing in some order of derivative). This change may be interpreted through the experimental data to indicate that the mode of strain adaptation to the compressive stress has changed or is changing. As part of the methods described herein, the experimental data may be interpreted and used to determine a compression threshold where one mode of strain adaptation for the tendon (or other soft tissue) changes or shifts to another mode of strain adaptation. The compression threshold may then be used by the methods described herein for performing a surgical method.

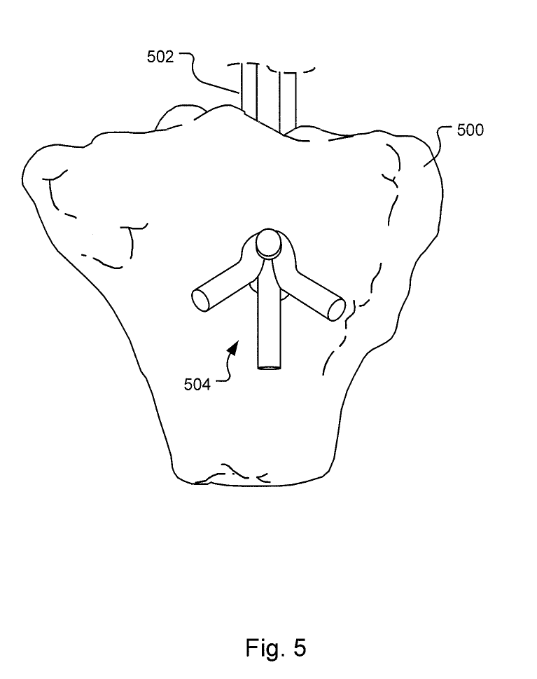

FIG. 5 is a schematic of a front view of an exemplary embodiment of a tendon bundle 502, 504 to be fixated on the upper extremity of the tibia 500 in an embodiment of an ACL repair. The tendon bundle has a portion 502 that exits the tibia 500 into the knee joint and toward the femur (not shown). This joint portion 502 of the tendon bundle replaces the natural ACL and may be tensioned in an ACL repair surgery to an appropriate level to replace the ACL (e.g., in function) in a patient.

In an exemplary embodiment of an ACL repair surgery, the free end of the tendon bundle 504 may be pulled to supply the appropriate tension in the joint portion 502 of the tendon bundle after the tendon bundle is fixed within the femur. The portion of the tendon bundle may be fixed within the tibia in such a manner that the tension in the joint portion 502 is maintained. As tension in the joint portion 502 is a factor in the success of an ACL repair, a surgeon may desire to fix the tendon bundle in the tibia 500 without significantly modifying the tension in the joint portion 502. The methods and devices described herein may be used to achieving this goal.

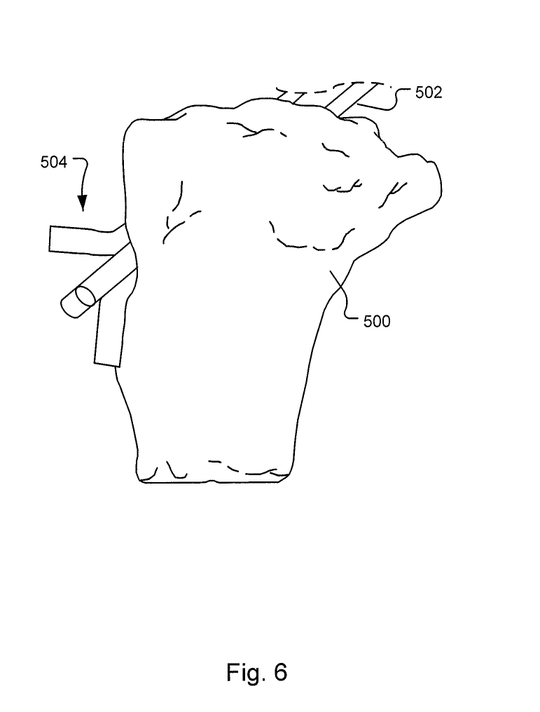

FIG. 6 is a schematic of a lateral view of an exemplary embodiment of a tendon bundle 502, 504 to be fixated on the upper extremity of the tibia 500 in an embodiment of an ACL repair.

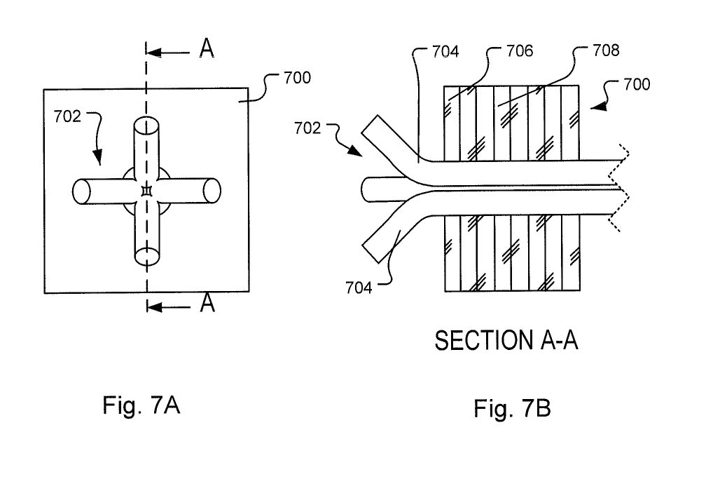

FIG. 7A is schematic front view of a tendon bundle 702 in a bone 700, showing the placement of a cross-section A-A. FIG. 7B is a schematic cross-section A-A view of the tendon bundle 702 in the bone 700. The tendon bundle 702 is shown in an exemplary embodiment as including four separate tendons 704, however other configurations of tendon bundles or a single tendon may be substituted. The cross-section of bone 700 shows different portions 706 and 708 within the bone, thereby generally indicating different portions of bone, such as cortical and cancellous bone. As different portions of bone may respond differently to the pressures of fixing soft tissue within the bone, the methods and devices described herein may account for these different responses and control and position fixation pressures accordingly.

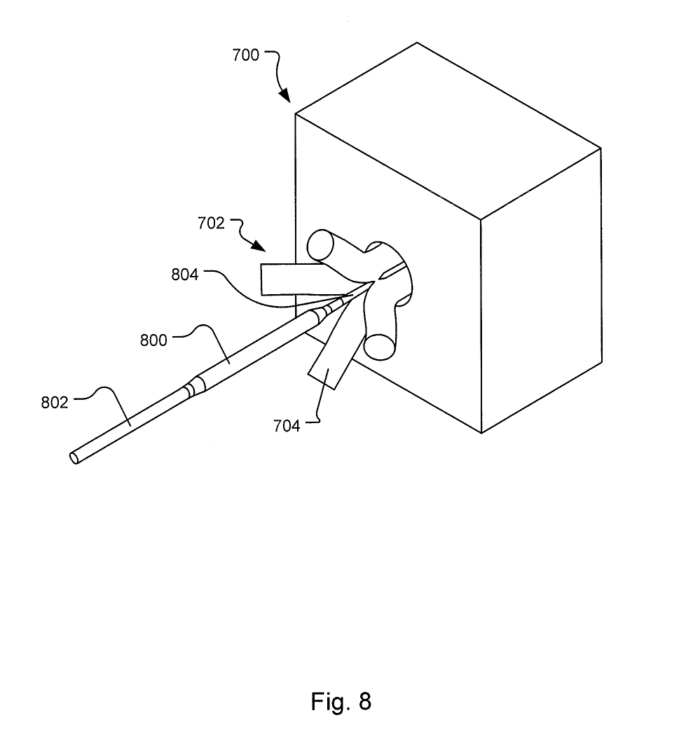

FIG. 8 is a perspective schematic view of an exemplary embodiment of an expandable member 800 being inserted into the bone 700 with the tendon bundle 702. The expandable member 800 is inserted along a guide wire 804. The expandable member has an expandable portion 800 that contains an inflatable body into which fluid may be inserted through a fluid conduit 802, thereby causing the inflatable body to inflate and the expandable member to expand. The fluid may be a relatively compressible fluid such as nitrogen gas, or may be a relatively non-compressible fluid such as saline water.

The expandable member 800 is shown in a non-expanded state. The guide wire 804 may be used to position the expandable member 800 within the bone, with the tendon bundle 702 placed between portion(s) of the expandable member 800 and the bone 700. The expandable member 800 as shown in its non-expanded state may be adapted to be positioned within the bone 700 in an ACL without significant forces applied to the tendon bundle that drive the tendon bundle into the joint (e.g., axial component parallel to guide wire 804) and/or without significant effect on the tension of a joint portion of the tendon bundle. As described further herein, the expandable member 800 may be expanded and the tendon bundle 702 fixed within the bone 700 without significant effect on the tension of the tendon bundle.

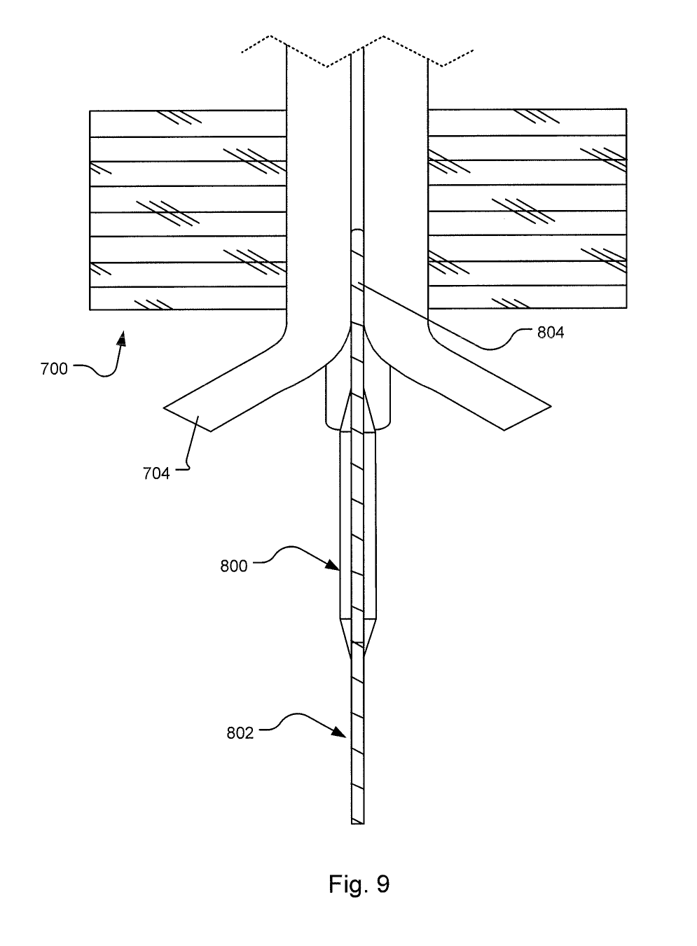

FIG. 9 is a schematic cross-section A-A view of the expandable member being inserted into the bone. The guide wire 804 may be positioned within the bone 700 with one or more of the tendon(s) 704 positioned between the guide wire 804 and the bone 700. The low profile of the expandable member 800 may be designed to fit within the bone with little or no disruption of the tendon(s) from their position. The fluid conduit 802 may also be designed to allow the passage of fluid into an inflatable body of the expandable member 800 in a controlled manner (e.g., without expanding or rupturing due to the pressure of the fluid) that allows the expandable member to be expanded to a controllable and/or known shape.

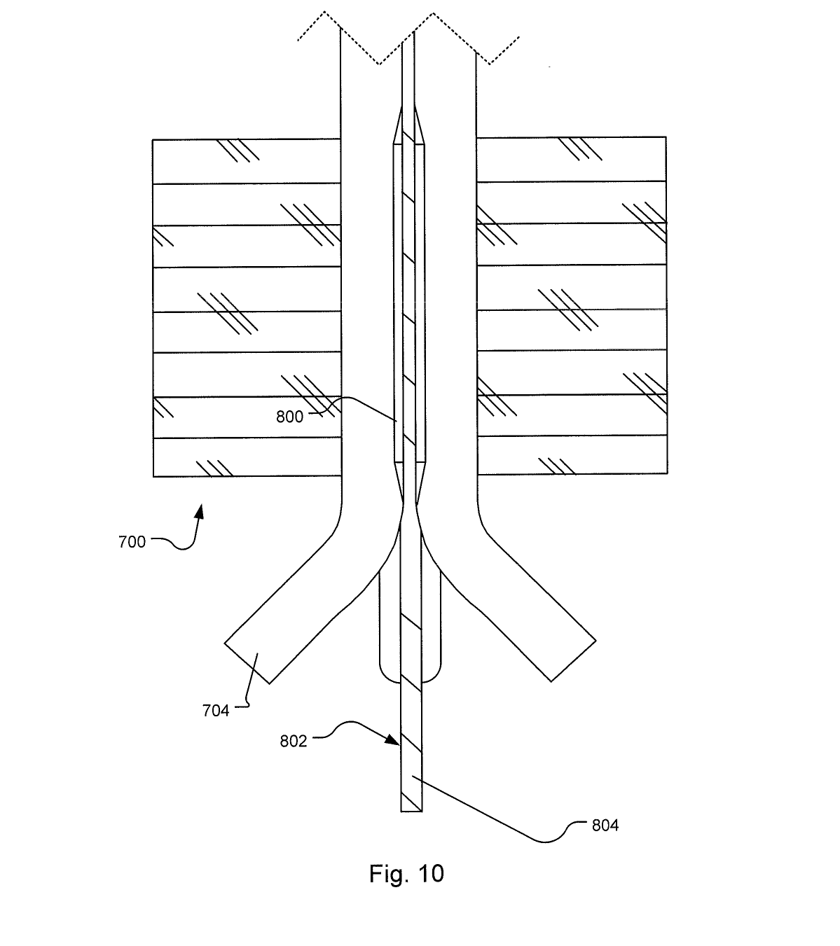

FIG. 10 is a schematic cross-section A-A view of the expandable member 800 positioned within the bone 700 and contacting the tendons 704 of the tendon bundle. The guide wire 804 may be used to position the expandable member 800 along the axis of the guide wire within the bone 700 at a position to compress the tendon(s) 704 against the bone via expansion of the expandable member. As described further herein, the fluid conduit 802 allows fluid to be inserted under pressure into an inflatable body within the expandable member 800, thereby confining/controlling the expansion of the expandable member to a desired position and extent.

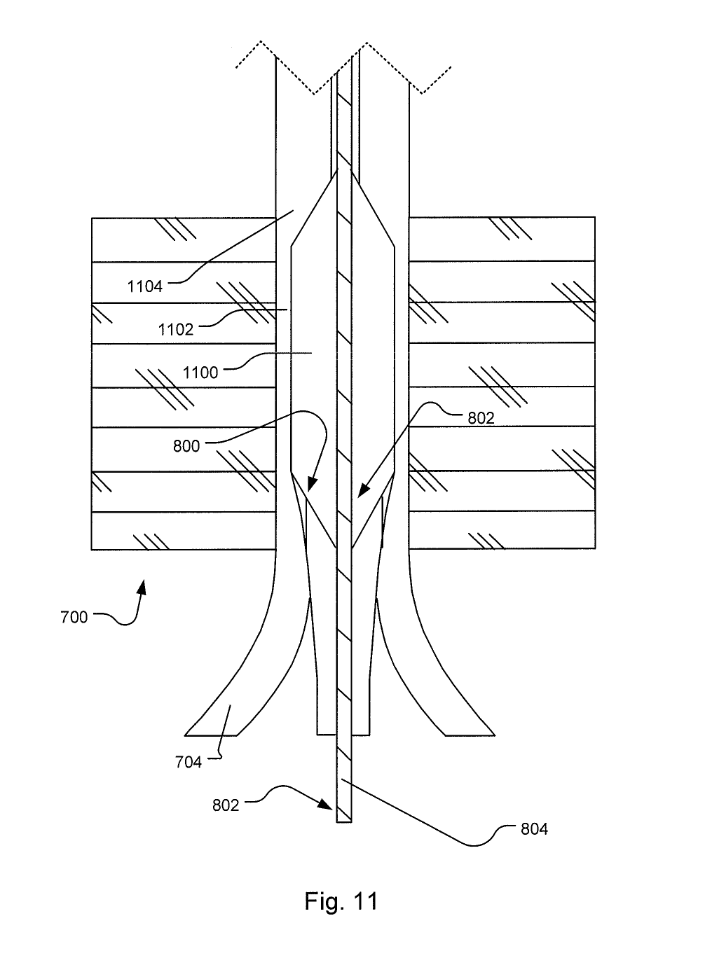

FIG. 11 is a schematic cross-section A-A view of the expandable member 800 in an exemplary expanded state. The expanded state may be designed to compress certain tendon portion(s) 1102 more than other tendon portion(s) 1104. In addition, due to varying attributes of soft tissues and/or bone, including local variations in tendon and/or bone properties, expandable members designed for uniform compression of soft tissue may reach a non-uniform equilibrium with the tendon(s) and/or bone. The expandable member may be designed/selected with these variations in mind in order to produce a desired compression for the surgical methods described further herein. The expandable member 800 may be designed to interface with the guide wire 804 while expanding to maintain a desired position within the bone 700 and relative to the tendon(s) 704 while the tendon(s) are compressed against the bone.

In an exemplary embodiment of the expandable member 800, the inflatable body creates a cavity 1100 when filled with pressurized fluid, as described further herein, through the fluid conduit 802. As described further herein, the expansion of expandable member 800 may be controlled during a surgical method. For example, the pressure of the fluid inserted into the inflatable body to create the cavity 1100 and compress the tendon(s) 704 may be controlled while measuring the compression applied to tendon(s). Additionally, a threshold compression may be determined to have been met through calculations relating to the controlled pressure and known properties of the expandable member, tendon, and/or bone.

The expandable member may further be designed to control the nature of forces applied to the tendon. For example, the expandable member may be designed to apply radial forces with respect to the axis of the guide wire 802. Based on the geometry of the cavity in the bone, the compressive forces applied to the tendon(s) 704 may be controlled to be substantially normal to the bone surface and/or normal to the outer surface of the expandable member. As described further herein, the expandable member and bone cavity can be adapted to compress the tendon while limiting forces along the axis of the guide wire, and therefore limiting any adverse effects on tension of the tendon(s).

In one embodiment, a reinforcing structure may be inserted/installed into the cavity 1100 as part of fixing the tendon(s) 704 within the bone. The reinforcing structure can be used within the expandable member as an embodiment of a fixation device to fix the tendon(s) to the bone. For example, the cavity 1100 may be filled with a fluid bone cement that may be hardened or set in order to fix the tendon(s) 704 in the bone 700 via maintaining the expandable member 800 in an expanded state and maintaining the compression of tendon(s) between the expandable member and the bone. The expandable member may be designed to fix the tendons to the bone via, for example, exterior surface treatment of the expandable member or other design of the interface with the tendon. Additionally, the expandable member and/or the inflatable body thereof may be designed to withstand/transmit compressive forces over a period of time appropriate for fixation of the tendon.

Multiple further embodiments described further herein may incorporate an expandable member filled and/or expanded by a fluid useful for fixing the tendon to the bone. For example, a first fluid may be inserted to compress the tendon (e.g., saline water) and a second fluid (e.g., bone cement) may be used to maintain the compression and fix the tendon. As another example, the same fluid (e.g., bone cement) used to expand the expandable member and compress the tendon may be used to fix the tendon. The compression on the tendon may be maintained while fluids are exchanged (e.g., removed and inserted) within an inflatable body of the expandable member and/or while a fluid is hardening. Alternatively, compression provided by the expandable member may be decreased or increased, as described further herein.

In another embodiment, the expandable member may be retracted (e.g., via deflating the inflatable body) and removed from the bone.

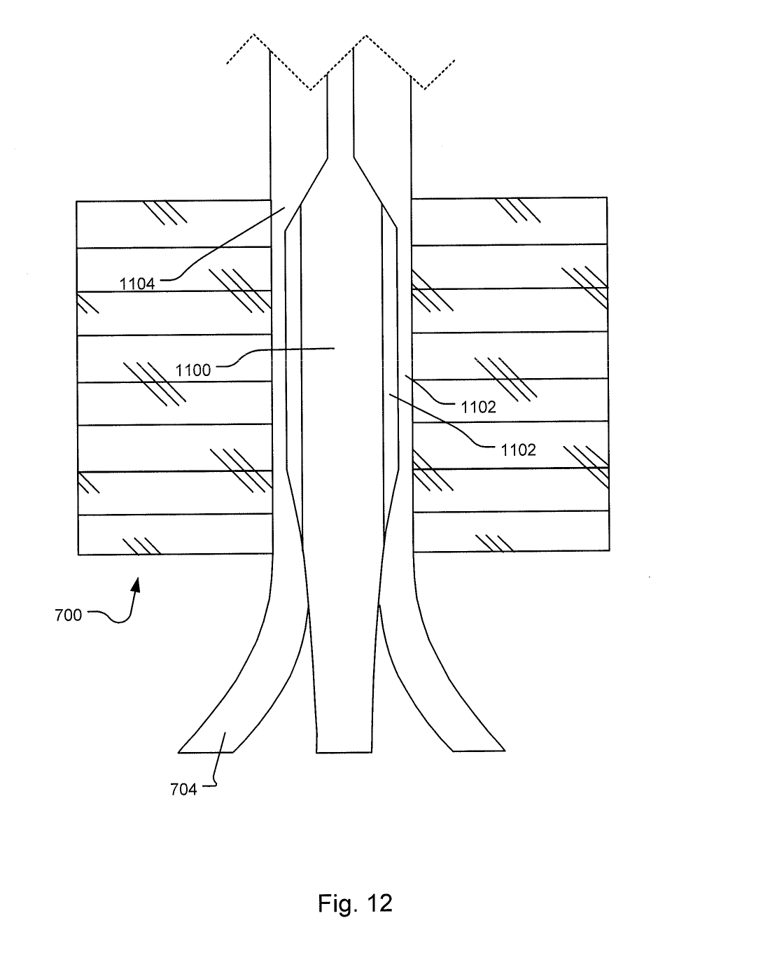

FIG. 12 is a schematic cross-section A-A view of the bone and tendon bundle after the expandable member has been retracted from its exemplary expanded state and removed from the bone. In the embodiment shown, the expandable member has been retracted and the tendon has been left in its compressed state. The tendons have not recoiled from their compressed states 1102 and 1104 with the decrease in compression from the expandable member or with the removal of the expandable member. In other embodiments, the tendons (or other soft tissue) may exhibit some recoil, based on the compression threshold attained or the particular properties of the soft tissue or mode(s) of strain adaptation to the compressive stresses applied. For example, the cavity 1100 may be modified based on recoil of the soft tissue and/or removal of the expandable device.

In the configuration shown, the tendons 704 inside the bone may accept a fixation device with limited axial movement within the bone tunnel. The shape of the expandable member may be designed, as described further herein, based on the shape of the fixation device and based on any expected/calculated modification of the cavity 1100 (e.g., recoil of the soft tissue) after the compression of the expandable member is removed in order to limit axial movement of the soft tissue while the fixation device is inserted/installed. The fixation device may create fixation-appropriate forces including compressive forces radially on the tendon(s), thereby fixing the tendon(s) to the bone with limited axial movement and/or effect on axial tension of the tendon(s).

What is claimed is:

1. A method comprising:
inserting a soft tissue into a bone recess formed in a bone;
inserting an expandable member into the bone recess;
expanding the expandable member thereby causing a compressive strain of the soft tissue against the bone in the bone recess;
measuring the compressive strain applied to the soft tissue; and
limiting the compressive strain to a predetermined value.

2. The method of claim 1, wherein the measuring step creates a measurement, and wherein the limiting step is based on the measurement.

3. The method of claim 1, wherein limiting comprises limiting a strain rate imparted to the soft tissue, and wherein the predetermined value is a predetermined strain rate.

4. The method of claim 1, further comprising:
decreasing a compressive force applied to the soft tissue;
removing the expandable member from the bone recess; and
inserting a bone fixation device against the soft tissue.

5. The method of claim 1, further comprising:
installing a reinforcing structure into the expandable member.

6. The method of claim 5, further comprising:
maintaining a compressive force applied to the soft tissue while performing the step of installing the reinforcing structure.

7. The method of claim 5, further comprising:
removing a material from the expandable member before installing the reinforcing structure.

8. The method of claim 5, further comprising:
removing a material from the expandable member while installing the reinforcing structure.

9. A bone fixation system, comprising:
an expandable member configured to fit within a bone recess along with a soft tissue;
an expansion control device adapted to limit to predetermined level a compressive strain value of the soft tissue between the expandable member and bone in the bone recess; and
a fixation device adapted to fix the soft tissue to the bone in the bone recess and adapted to fit within the bone recess with the soft tissue.

10. The system of claim 9, wherein the fixation device is further adapted to fit within the bone recess with the soft tissue after the soft tissue has been compressed by the expandable member.

11. The system of claim 9, wherein the compressive strain value of the soft tissue is a level of compressive strain between the expandable member and the bone recess.

12. The system of claim 9, wherein the compressive strain value of the soft tissue is a strain rate of the soft tissue.

13. A bone fixation system, comprising:
an expandable member configured to fit within a bone recess formed in a bone along with a soft tissue;
an expansion control device adapted to limit to a predetermined value a compressive strain value of the soft tissue between the expandable member and the bone in the bone recess; and
a reinforcing structure adapted to fix the soft tissue to the bone in the bone recess adapted to be inserted within the expandable member.

14. The system of claim 13, wherein the reinforcing structure is adapted to be inserted in the expandable member as a fluid and to be hardened within the expandable member.

15. The system of claim 13, wherein the compressive strain value of the soft tissue is a level of compressive strain between the expandable member and the bone recess.

16. The system of claim 13, wherein the compressive strain value of the soft tissue is a strain rate of the soft tissue.
